SEQUENCE LISTING (8 sequences)

SEQ ID NO: 1 - DNA, 1003 bp, Zea mays L
aacagttagaaaatacactactgctaggctcatgccgatcttgatcattacatgtacatatgttgcattatattattcactgcgagctatatatatgtctcctgctctctctttcaaacgatatacaccttttgcacttgttcaaccgttgcactccactgaaaccgtactagaagtttctttttgaccgcccttttttccctattctattgcaggatgcttccattgctcacccccaagagacagaatggccttacacgtactggccacctgacaaccaagatcatcatgggtagtagcaacagggacctggtaaatagtggcgatgtgaatgctgaaggaacactaaccgcaaccactttcactggaatggtttttgtctgcttgaatatgcagcgttgctttcttggagaatgtacacctacaatagtggggaaagaacagagaaaaaaaacgtatctctgtagatctccctcaataaagaaatgaggataaaactagttaacagtgtgagctctccaagacagaagagacaaaatggccaacgaaaagagaagggggaaagaccgatgggaggaaaggtactgtgtgtgctgacattgaagggatcatgtgtggacagtttagatgtattagaacaccttaatcaagtataactataatcagtttacggtttatcacagttttttattcgtgctacctgttagtggcattgtttgcatgtacattaacccttttaatttcttttagtttctaaattgatgctgattttcctttggttagttcatgctaatatgcatcacgaagaaataaagttagtaagaagctcataattccttcctttttttagcacaaacattgtttatttattgttttattgttattgttgatgtaggttttttgcaagtagaatacaagatatactgttcaaggaataagttgattcatatgatcattgttaaacacatagagatatttaaagaaataatatcataacataagt

SEQ ID NO: 2 - DNA, 1027 bp, Zea mays L
aacagttagaaaatacactactgctaggctcatgccgatcttgatcattacatgtatatatgttgcattatattattcactgcgagctatatatatgtctcctgctctctctttcaaacgatatatacctttttgcacttgttcaaccgttgcactccactgaaaccgtactagaagtttctttttgaccgtccttttttccctattctattgcaggatgcttccattgctcacccccaagagacagaatggccttacccgtactggccacctgacaaccaagatcatcatgggtagtagcaacagggacctggtaaatagtggcgatgatgtgaatgctgaaggaacactaaccgcaaccactttcactggaatggtttttgtctgcttgaatatgcagcgttgctttcttggagaatgtacacctacaatagtggggaaagaacagagaaaaaaaacatatctctgtagatctccctcaataaagaaatgaggataaaactagttaacagtgtgagctctccaagacagaagagacaaaatggccaacgaaaagagaaggggggaaagaccgatgggaggaaaggtactgtgtgtgctgacattgaaggggtcatgtgtggaccgttagatgtattagagcaccttaatcaagtataactataatcagtttacagtttatcacagtttttttattcgtactacctgttttccaaatgtaaaaactagtggcattgtttgcatgcatgtgcattaacccttttaatttctttcagtttctaaattgatgctgattttcttttggttagttcatgctaatatgcatcacgaagaaataaacatagtaagaagctcgtaattccttcctttttagcacaaacattttttatttattattttattgttattattgatgtaggttttttgcaagtagaattcaagctatactgttcaaggaataagttgattcatatgatcattgtcaatcacatagagatatttaaacagataatatcataacataagt

SEQ ID NO: 3 - DNA, 20 bp, Artificial Sequence
  The sequence is synthesized.
ggctttaagcaaggccaacg

SEQ ID NO: 4 - DNA, 21 bp, Artificial Sequence
  The sequence is synthesized.
gctcactagcaaggcttctca

SEQ ID NO: 5 - DNA, 23 bp, Artificial Sequence
  The sequence is synthesized.
gccggcttggaagaactgataat

SEQ ID NO: 6 - DNA, 25 bp, Artificial Sequence
  The sequence is synthesized.
ccctgttgctactacccatgatgat

SEQ ID NO: 7 - DNA, 1527 bp, Zea mays L
atggccgttctcgtcttcctggcgatcgccctggttagcgcagcaagacggagaaacaagaaccatccctccccctaccgctgctactcatgcacgcgcgcactgcctcctggtgccgccgcgtatgatccagacatgaatagggcgcacagcagcagctccaagctcatcaacgagaagctcgtggaacaccggatctccactgcgaagcactgcccccactgcggcgagaaaatcgacagcaaaccgttatgcacctcctcagccagcatgagacctggtggtggtgcaacttgttttgatgggattaattgctgtaactgcaagaaggattgggtggggctgccggcaggcgtcaagttcgatccaactgaccaggagctgatcgagcacctcgaggcgaaagtgaaggatgaaggctcgagatctcaccctctcatcgacgagttcatacccacaatagatggggaggacggcatatgttacacccaccccgagaaacttccaggtgtgacgagggatggcctaagcaagcacttcttccaccggccgtccaaggcctacacgacgggcacgaggaagaggaggaagatacagacggagtgcgacgtccacaagggggagacgaggtggcacaagaccggcaagacgcggccggtgatggtgaacggccggcagaaggggtgcaagaagatattggtgctgtacaccaacttcggcaagcaccgcaagccggagaagacgaactgggtcatgcaccagtaccacctcggcgacctagaagaggagaaggagggggagctggtggtgtgcaagatcttctaccagacgcaacccaggcagtgcagctggtcctccgaccggggcgccgccgccactgccttggcagcaacggcggcagcggcggcggcgcaggagcagcataggagggatagtggcagtggcagctgctcgtctagggaccacgaggtgtcagccacatcgttcccagccggatacacagtcaccacggccgtcgagatgcagcagcatatgaagcagcctgcggaccatttcagcttcgcgcctttcaggaaaaccttcgaccaggaggttggtataggtggtgatcaggtgccatctaatcagcttggacgttcagagccgcatcatgccggcctggggcagcagccacacggcccggtgctcgcgacgaagacggctgtgcctgctacagctttcctgatcagtaggccatcgaaccctgtctcgactatagtgccacctgcaatgcagcacgcatcagttgttctcgatcatgatcagttccatgtgccagcaatccttctccatcaccatgacaaatttcagaacatgcaccaacagccacaacaaaagcttgaccgcagatctgccggcttggaagaactgataatgggctgcacgtcgtccacaagcacaaaaggagatgcttccattgctcacccccaagagacagaatggccttacacgtactggccacctgacaaccaagatcatcatgggtag

SEQ ID NO: 8 - protein, 508 aa, Zea mays L
MAVLVFLAIALVSAARRRNKNHPSPYRCYSCTRALPPGAAAYDPDMNRAHSSSSKLINEKLVEHRISTAKHCPHCGEKIDSKPLCTSSASMRPGGGATCFDGINCCNCKKDWVGLPAGVKFDPTDQELIEHLEAKVKDEGSRSHPLIDEFIPTIDGEDGICYTHPEKLPGVTRDGLSKHFFHRPSKAYTTGTRKRRKIQTECDVHKGETRWHKTGKTRPVMVNGRQKGCKKILVLYTNFGKHRKPEKTNWVMHQYHLGDLEEEKEGELVVCKIFYQTQPRQCSWSSDRGAAATALAATAAAAAAQEQHRRDSGSGSCSSRDHEVSATSFPAGYTVTTAVEMQQHMKQPADHFSFAPFRKTFDQEVGIGGDQVPSNQLGRSEPHHAGLGQQPHGPVLATKTAVPATAFLISRPSNPVSTIVPPAMQHASVVLDHDQFHVPAILLHHHDKFQNMHQQPQQKLDRRSAGLEELIMGCTSSTSTKGDASIAHPQETEWPYTYWPPDNQDHHG

(19) United States
(12) Patent Application Publication
(10) Pub. No.: US 2022/0396805 A1
(43) Pub. Date: Dec. 15, 2022

(54) DNA SEQUENCE FOR REGULATING MAIZE LEAF ANGLE, AND MUTANT, MOLECULAR MARKERS, DETECTION PRIMERS, AND USE THEREOF

(71) Applicants: SOUTH CHINA AGRICULTURAL UNIVERSITY, Guangzhou (CN); BIOTECHNOLOGY RESEARCH INSTITUTE, CHINESE ACADEMY OF AGRICULTURAL SCIENCES, Beijing (CN)

(72) Inventors: Haiyang WANG, Guangzhou (CN); Baobao WANG, Beijing (CN); Yurong XIE, Beijing (CN); Xing LI, Beijing (CN); Bingbing ZHAO, Beijing (CN); Yongping ZHAO, Beijing (CN)

(73) Assignees: SOUTH CHINA AGRICULTURAL UNIVERSITY, Guangzhou (CN); BIOTECHNOLOGY RESEARCH INSTITUTE, CHINESE ACADEMY OF AGRICULTURAL SCIENCES, Beijing (CN)

(21) Appl. No.: 17/775,294

(22) PCT Filed: Oct. 21, 2020
(86) PCT No.: PCT/CN2020/122375

§ 371 Date: May 8, 2022

(30) Foreign Application Priority Data
Jan. 22, 2020 (CN) 202010075654.6

(51) Int. Cl. C12N 15/82; C07K 14/415

(52) U.S. Cl. CPC C12N 15/8262; C07K 14/415

(57) ABSTRACT

A key DNA sequence for regulating a maize leaf angle and a mutant thereof are provided, which have polynucleotide sequences shown in SEQ ID No. 1 and SEQ ID No. 2, respectively. The DNA sequence for regulating a maize leaf angle and the mutant thereof provided by present disclosure can regulate the expression of ZmNAC16 gene in a maize pulvinus, and thus can be used for the improvement of maize leaf angle and plant type and further for the cultivation of new maize varieties. The present disclosure further provides specific detection primers for detecting mutations of the DNA key sequence and the mutant, and detection primers for detecting an expression level of ZmNAC16 gene in maize. These detection primers can be used to directionally improve a maize leaf angle and also shows application potential for breeding of dense-planting-tolerant and high-yield maize.

### TECHNICAL FIELD

The present disclosure relates to a DNA sequence for regulating a maize leaf angle and a mutant thereof, and particularly to mutants and detection primers for an intron and a 3′-UTR region that regulate the expression of a gene ZmNAC16. The present disclosure further relates to use of the aforementioned fragments in the regulation of a maize leaf angle, and belongs to the field of molecular breeding of maize.

### BACKGROUND

As one of the most important crops in China, maize plays an important role in the food security and agricultural development of China. However, due to the limitation of an arable land area (the China's arable land area only accounts for 7% of the world's arable land area, but needs to feed about 20% of the world's population), it is necessary to increase a yield per unit area to increase a maize output of China. Moreover, an average maize output in China is only 60% of that in the United States, and thus there is huge potential and room for improvement.

Studies have shown that improving the dense planting tolerance and planting density of a crop variety is the key to increasing a maize yield per unit area. Over the past 80 years, a maize planting density in the United States of America has increased from about 30,000 plants/ha. in the 1930s to about 70,000 plants/ha. at present (62,000 to 104,000 plants/ha.); a maize yield per unit area has also increased from 1,287 kg/ha. in the 1930s to 9,595 kg/ha. in 2010 (USDA-NASS, 2012); and the yield per plant and heterosis of maize have not been significantly improved, and the increase in the maize yield per unit area is largely caused by the continuous improvement of the dense planting tolerance and planting density of a crop variety. Research on maize varieties of different years in China has also shown that, compared with early varieties, modern varieties tend to have higher dense planting tolerance in terms of photosynthetic efficiency, lodging resistance, hollow stalk rate, and yield. Therefore, improving the dense planting tolerance and planting density of a crop variety is an important goal and trend in modern maize breeding and production.

Reducing a leaf angle of plant is the key to improving the dense planting tolerance of maize. A small leaf angle can minimize the mutual shading among maize plants, improve the overall canopy structure of a field, enhance the ventilation and light transmission among plants, and facilitate maize functional leaves (ear leaf, first leaf above an ear, and first leaf below an ear) to capture sunlight and conduct photosynthesis, which is conducive to high maize yield. Moreover, excellent ventilation and light transmission will greatly increase a ratio of red light/far-red light (R/FR) in a lower layer of a plant, and reduce the adverse effects of excessive stem growth, root system weakening, and stem strength reduction caused by dense planting and shade avoidance reactions, which is conducive to stable maize production. In addition, studies have shown that a small leaf angle is also conducive to the nitrogen assimilation of leaves to promote grain filling, which directly affects the maize yield. Studies of different institutions all have shown that a leaf angle is becoming smaller and smaller in the modern maize breeding process, and this trend is highly related to the improvement of dense planting tolerance of maize, which also reflects the importance of a leaf angle for density-tolerant maize breeding from another perspective.

Studies have shown that reported genes for regulating the leaf angle change in maize mainly include genes from gene families such as SPL, LOB, MYB, bZIP, and Homeodomain-like, such as LG1, LG2, LG3, LG4, LGN1, DWIL1, DRL1, DRL2, ZmRAVL1, qLA1, ZmCLA4, ZmTAC1, BRD1, ZmBRL2, ZmBRL3, ZmBRHb, ZmBRL1, and ZmBR11a. However, so far, there has been no report that NAC family genes (NAC domain containing protein) regulate the maize leaf angle phenotype. In addition, frameshift, early termination, or other mutations causing protein function changes of the above-mentioned genes for regulating the leaf angle change often lead to a drastic change in the leaf angle, and are often accompanied by adverse effects such as hindered inflorescence development, abnormal leaf organ development, and sharp yield reduction, which cannot be directly applied to the practice of maize breeding. In contrast, some natural mutations in non-coding regions often only change an expression level of a gene, and bring few adverse effects. In particular, some natural mutations retained through long-term breeding selection show promising application prospects in breeding. Therefore, the accurate identification of these natural mutations in non-coding regions has important application potential for molecular breeding of maize.

### SUMMARY

A first objective of the present disclosure is to provide a key DNA sequence for regulating a maize leaf angle.

A second objective of the present disclosure is to provide a mutant of the DNA sequence for regulating a maize leaf angle.

A third objective of the present disclosure is to provide molecular markers for regulating the expression of ZmNAC16 gene in a maize pulvinus.

A fourth objective of the present disclosure is to provide specific detection primers for detecting a mutation of the key DNA sequence for regulating a maize leaf angle or the mutant thereof.

A fifth objective of the present disclosure is to provide detection primers for detecting an expression level of ZmNAC16 gene in maize.

The above objectives of the present disclosure are achieved by the following technical solutions.

The present disclosure first provides a key DNA sequence for regulating a maize leaf angle, where the DNA sequence has a polynucleotide from the group consisting of (a), (b), (c), and (d):

(a): a polynucleotide sequence shown in SEQ ID No. 1;

(b): a polynucleotide sequence that can hybridize with a complementary sequence of SEQ ID No. 1 under stringent hybridization conditions;

(c): a polynucleotide that has at least 90% or more homology with the polynucleotide shown in SEQ ID No. 1; and

(d) a mutant obtained through deletion, substitution, or insertion of one or more bases on the basis of the polynucleotide shown in SEQ ID No. 1, where the mutant still has a function or activity of regulating a maize leaf angle.

The key DNA sequence for regulating a maize leaf angle provided by the present disclosure can regulate the expression change of the ZmNAC16 gene in a maize pulvinus.

As a preferred embodiment of the mutant, the mutant is obtained through the change from T to C at SNP_3_6945310_C/T, the 1 bp base insertion at Indel_3_6945248_C/CT, or the 4 bp base deletion at Indel_3_6945836_T/TTGCA, and has a polynucleotide sequence shown in SEQ ID No. 2; and the mutant can regulate the expression of the ZmNAC16 gene in a maize pulvinus, which makes a leaf angle reduced, but does not bring unfavorable phenotypes. Therefore, SNP_3_6945310_C/T, Indel_3_6945248_C/CT, and Indel_3_6945836_T/TTGCA can be used as molecular markers for regulating the expression of the ZmNAC16 gene in a maize pulvinus.

The present disclosure further provides detection primers for detecting mutations of SNP_3_6945310_C/T, Indel_3_6945248_C/CT, and Indel_3_6945836_T/TTGCA. As a preferred embodiment, the detection primers may have nucleotide sequences shown in SEQ ID No. 3 and SEQ ID No. 4, respectively.

The specific detection primers can be used to detect the mutations of SNP_3_6945310_C/T, Indel_3_6945248_C/CT, and Indel_3_6945836_T/TTGCA in maize varieties, thereby achieving marker-assisted selection (MAS) of maize. In addition, those skilled in the art can design specific detection primers according to SEQ ID No. 1 or SEQ ID No. 2, or design primers for detecting the mutations of SNP_3_6945310_C/T, Indel_3_6945248_C/CT, and Indel_3_6945836_T/TTGCA according to a conventional method in the art, which are also within the protection scope of the present disclosure.

The present disclosure further provides specific amplification primers for detecting an expression level of the ZmNAC16 gene, and the specific detection primers have nucleotide sequences shown in SEQ ID No. 5 and SEQ ID No. 6, respectively. The specific detection primers can be used to detect an expression level of the ZmNAC16 gene in maize varieties, thereby providing a reference for maize breeding.

A coding region of the ZmNAC16 gene in the present disclosure has a polynucleotide sequence shown in SEQ ID No. 7, and a protein encoded thereby has an amino acid sequence shown in SEQ ID No. 8.

Further, the present disclosure provides a method for cultivating a new high-yield or dense-planting-tolerant maize variety, including: increasing an expression level of the ZmNAC16 gene in a maize pulvinus to reduce the maize leaf angle and improve the dense planting tolerance of maize. Therefore, all methods to reduce the maize leaf angle and improve the dense planting tolerance of maize by increasing the expression level of the ZmNAC16 gene in the maize pulvinus belong to the protection scope of the present disclosure. The methods include a method of using an expression cassette constructed from another constitutive or tissue-specific promoter and ZmNAC16 to drive the high expression of the ZmNAC16 gene in the maize pulvinus, or using another natural mutation to achieve the high expression of the ZmNAC16 gene in the maize pulvinus.

Furthermore, the present disclosure provides an expression cassette carrying the DNA sequence shown in SEQ ID No. 1 or the mutant of the DNA sequence shown in SEQ ID No. 2, a plant recombinant expression vector carrying the expression cassette, and a transgenic cell line, and host bacteria.

The plant recombinant expression vector is a plant recombinant expression vector constructed from the expression cassette and a plasmid or expression vector, which can transfer the expression cassette into a plant host cell, tissue, or organ.

The DNA sequence or the mutant thereof according to the present disclosure can be used to prepare a transgenic plant. For example, the plant recombinant expression vector carrying the DNA sequence or mutant thereof is transformed into a plant cell, tissue, or organ through Agrobacterium tumefaciens (A. tumefaciens) mediation, particle bombardment, and other methods, and then the transformed plant cell, tissue, or organ is cultivated into a plant to obtain a transgenic plant. The starting vector used to construct the plant expression vector can be any binary vector for A. tumefaciens-mediated transformation of a plant or a vector that can be used for plant microprojectile bombardment.

In order to implement the present disclosure, conventional compositions and methods for preparing and using plant expression vectors and host cells are well-known to those skilled in the art, and specific methods can refer to Sambrook and the like, for example.

The plant recombinant expression vector may also carry a selective marker gene for selecting transformed cells. The selective marker gene is provided to select transformed cells or tissues. The marker gene includes: a gene conferring antibiotic resistance, a gene conferring herbicide resistance, and the like. In addition, the marker gene also includes a gene of a phenotypic marker, such as β-galactosidase and fluorescent protein.

In conclusion, the key DNA sequence for regulating a maize leaf angle and the mutant thereof, the molecular markers and the specific detection primers for detecting the mutations of the molecular markers, and the specific detection primers for detecting the expression level of ZmNAC16 gene provided by the present disclosure can be used to cultivate a new high-yield or dense-planting-tolerant maize variety, especially to improve a leaf angle, a plant type, and a yield of maize.

As reference, the present disclosure provides a method for regulating a maize leaf angle, including: using the DNA sequence shown in SEQ ID No. 1 or the mutant of the DNA sequence shown in SEQ ID No. 2 to regulate the expression of the ZmNAC16 gene in maize.

The transformation scheme and the scheme for introducing the polynucleotide or polypeptide into a plant in the present disclosure may vary according to a type of a plant (monocotyledon or dicotyledon) or plant cell for transformation. Suitable methods for introducing the polynucleotide or polypeptide into a plant cell include: microinjection, electroporation, A. tumefaciens-mediated transformation, direct gene transfer, high-speed ballistic bombardment, and the like. In a specific embodiment, various transient transformation methods can be used to provide the expression cassette of the present disclosure to a plant. Conventional methods can be used to generate stable transformed plants from transformed cells (McCormick et al. Plant Cell Reports. 1986. 5: 81-84).

The present disclosure can be used to transform any plant species, including but not limited to: monocotyledon or dicotyledon, preferably maize.

The DNA sequence for regulating a maize leaf angle or the mutant thereof provided by the present disclosure can regulate the expression of the ZmNAC16 gene in a maize pulvinus, which has important significance for improving the maize leaf angle and plant type, and can be further used for selective breeding of new maize varieties. The present disclosure further provides molecular markers SNP_3_6945310_C/T, Indel_3_6945248_C/CT, and Indel_3_6945836_T/TTGCA for regulating the expression of ZmNAC16 gene, specific detection primers for detecting the mutations of the molecular markers, and specific detection primers for detecting an expression level of the ZmNAC16 gene in maize, which can be directly used to directionally improve a maize leaf angle and also shows huge application potential for selective breeding of dense-planting-tolerant and high-yield maize varieties.

Definitions of Terms in the Present Disclosure

Unless otherwise defined, all technical and scientific terms used herein have the same meanings as commonly understood by those of ordinary skill in the art to which the present disclosure belongs. Although any methods, devices, and materials similar or equivalent to those described herein can be used in the practice or test of the present disclosure, preferred methods, devices, and materials are now described.

In the context of the present disclosure, the term “mutant” refers to a DNA sequence with a variation, in which one or more nucleotides are preferably deleted, added, and/or substituted while basically maintaining the original DNA sequence. For example, one or more base pairs can be deleted at the 5′ or 3′ end of a DNA sequence to produce a truncated DNA sequence; or one or more base pairs can also be inserted, deleted, or substituted within a DNA sequence. A variant DNA sequence or a part thereof can be produced by, for example, standard DNA mutagenesis or chemical synthesis to obtain a variant DNA sequence. Mutant polynucleotides also include synthetic polynucleotides, such as mutants obtained through site-directed mutagenesis, or mutants obtained through recombinant (such as DNA shuffling), or mutants obtained through natural selection.

The term “polynucleotide” or “nucleotide” refers to deoxyribonucleotide, deoxyriboside, riboside, or ribonucleotide and a polymer thereof in a single-stranded or double-stranded form. Unless otherwise specifically limited, the term encompasses nucleic acids containing known analogues of natural nucleotides, and the analogues have binding properties similar to a reference nucleic acid and are metabolized in a manner similar to that of natural nucleotides. Unless otherwise specifically limited, the term also refers to oligonucleotide analogues, including peptide nucleic acids (PNAs), and DNA analogues used in antisense technology (organothiophosphate, phosphoramidate, and the like). Unless otherwise specified, a specific nucleic acid sequence also implicitly encompasses conservatively modified mutants (including but not limited to degenerate codon substitutions) and complementary sequences thereof, and explicitly specified sequences. Specifically, a degenerate codon substitution can be achieved by generating a sequence in which one or more selected (or all) codons are subjected to position-3 substitution with mixed bases and/or deoxyinosine residues (Batzer et al., Nucleic Acid Res. 19: 5081 (1991); Ohtsuka et al., J. Biol. Chem. 260: 2605-2608 (1985); and Cassol et al., (1992); Rossolini et al., Mol Cell. Probes 8: 91-98 (1994)).

The term “homology” refers to a level of similarity or percent identity between polynucleotide sequences in terms of percent nucleotide position identity (namely, sequence similarity or identity). The term “homology” used herein also refers to a concept of similar functional properties between different polynucleotide molecules. For example, promoters with similar functions may have homologous cis-elements. When polynucleotide molecules can be specifically hybridized under specified conditions to form duplex molecules, the polynucleotide molecules are homologous. Under these conditions (stringent hybridization conditions), a polynucleotide molecule can be used as a probe or primer to identify another polynucleotide molecule with homology.

The “stringent hybridization conditions” in the present disclosure refers to low ionic strength and high temperature conditions known in the art. Generally, under the stringent conditions, a detectable degree of hybridization between a probe and a target sequence thereof is higher than a detectable degree of hybridization of the probe with other sequences (for example, at least 2 times more than the background). Stringent hybridization conditions are sequence-dependent and will be different under different environmental conditions, and long sequences are specifically hybridized at high temperatures. By controlling the stringency or washing conditions of hybridization, a target sequence that is 100% complementary to a probe can be identified. Detailed guidance on nucleic acid hybridization can refer to the relevant literature (Tijssen, “Techniques in Biochemistry and Molecular Biology-Hybridization with Nucleic Probes,” Overview of principles of hybridization and the strategy of nucleic acid assays. 1993). More specifically, the stringent conditions usually involve a temperature about 5° C. to 10° C. lower than a thermal melting point (Tₘ) of a specific sequence at a specified ionic strength and pH. Tₘ refers to a temperature at which 50% of a probe complementary to a target sequence is hybridized with the target sequence in an equilibrium state (at a specified ionic strength, pH, and nucleic acid concentration) (because the target sequence exists in excess, 50% of the probe is occupied in the equilibrium state at Tₘ). The stringent conditions can be as follows: a salt (sodium ion or other salts) concentration is lower than about 1.0 M (which is usually about 0.01 M to 1.0 M) at pH 7.0 to 8.3; and a temperature is at least 30° C. for short probes (including but not limited to 10 to 50 nucleotides), and at least about 60° C. for long probes (including but not limited to more than 50 nucleotides). The stringent conditions can also be achieved by adding a destabilizer such as formamide. For selective or specific hybridization, a positive signal can be at least twice the background hybridization, and optionally 10 times the background hybridization. Exemplary stringent hybridization conditions can be as follows: 50% formamide, 5×SSC, and 1% SDS, and incubation at 42° C.; or 5×SSC, 1% SDS, incubation at 65° C., washing in 0.2×SSC, and washing in 0.1% SDS at 65° C. The washing can be conducted for 5 min, 15 min, 30 min, 60 min, 120 min, or more.

In the present disclosure, the “more” usually refers to 2 to 8 and preferably 2 to 4; the “substitution” refers to the replacement of one or more amino acid residues with different amino acid residues; the “deletion” refers to the reduction in the number of amino acid residues, that is, the lack of one or more amino acid residues; and the “insertion” refers to the change in a sequence of amino acid residues, and relative to natural molecules, the change results in the addition of one or more amino acid residues.

The term “coding sequence” refers to a nucleic acid sequence that can be transcribed into RNA.

The term “plant promoter” refers to a natural or non-natural promoter that is functional in plant cells. Constitutive plant promoters function in most or all tissues throughout plant development. Any plant promoter can be used as a 5′ regulatory element to regulate the expression of one or more specific genes operably linked to the promoter. When operably linked to a transcribable polynucleotide molecule, the promoter generally causes the transcription of the transcribable polynucleotide molecule, and its transcription mode is similar to a transcription mode of a transcribable polynucleotide molecule that is usually linked to the promoter. Plant promoters may include artificial, chimeric or hybrid promoters produced by manipulating known promoters. Such promoters can also combine cis-elements from one or more promoters, for example, by adding heterologous regulatory elements to an active promoter with some or all regulatory elements itself.

The term “cis-element” refers to a cis-acting transcriptional regulatory element that confers overall control for gene expression. A cis-element can play the roles of binding to a transcription factor for regulating transcription and trans-acting a protein factor. Some cis-elements can each bind to more than one transcription factor, and a transcription factor can interact with more than one cis-elements through different affinities.

The term “operably linked” refers to the connection of a first polynucleotide molecule (such as a promoter) to a second transcribable polynucleotide molecule (such as a target gene), where the polynucleotide molecules are arranged such that the first polynucleotide molecule affects a function of the second polynucleotide molecule. Preferably, the two polynucleotide molecules are parts of a single contiguous polynucleotide molecule, and more preferably are adjacent. For example, if a promoter regulates or mediates the transcription of a target gene in a cell, the promoter is operably linked to the target gene.

The term “transcribable polynucleotide molecule” refers to any polynucleotide molecule that can be transcribed into an RNA molecule. A construct is introduced into a cell in such a way that a transcribable polynucleotide molecule can be transcribed into a functional mRNA molecule, and then the functional mRNA molecule is translated and thus expressed into a protein product. In order to inhibit the translation of a specific target RNA molecule, a construct capable of expressing an antisense RNA molecule can also be constructed.

The term “plant recombinant expression vector” refers to one or more DNA vectors to achieve plant transformation, and these vectors are often referred to as binary vectors in the art. Binary vectors and vectors with helper plasmids are mostly used for A. tumefaciens-mediated transformation commonly. Binary vectors usually include cis-acting sequences required for T-DNA transfer, selective markers engineered to be expressed in plant cells, heterologous DNA sequences to be transcribed, and the like.

The term “transformation” refers to a method of introducing a heterologous DNA sequence into a host cell or organism.

The term “expression” refers to the transcription and/or translation of an endogenous gene or transgene in a plant cell.

The term “recombinant host cell strain” or “host cell” refers to a cell with the polynucleotide of the present disclosure, regardless of the method used for insertion to produce a recombinant host cell, such as direct uptake, transduction, f-pairing, or other methods known in the art. The exogenous polynucleotide can be maintained as a non-integrated vector such as a plasmid or can be integrated into a host genome. The host cell can be a prokaryotic cell or a eukaryotic cell, and the host cell can also be a monocotyledonous or dicotyledonous cell.

### BRIEF DESCRIPTION OF THE DRAWINGS

FIG. 1A shows the significant correlation of the three variation sites in the ZmNAC16 intron and 3′-UTR region with the flowering period and leaf number phenotype of maize obtained by the genome-wide association study (GWAS) method, where the three mutation sites are SNP_3_6945310_C/T, Indel_3_6945248_C/CT, and Indel_3_6945836_T/TTGCA and the red arrow parallel to the X-axis in the figure represents ZmNAC16; and FIG. 1B shows the gene structure of ZmNAC16 and the location information of the three mutation sites SNP_3_6945310_C/T, Indel_3_6945248_C/CT, and Indel_3_6945836_T/TTGCA.

FIG. 2 shows the mutations of 16 different groups of inbred lines at the three mutation sites (SNP_3_6945310_C/T, Indel_3_6945248_C/CT, and Indel_3_6945836_T/TTGCA), where overall, the three sites SNP_3_6945310_C/T, Indel_3_6945248_C/CT, and Indel_3_6945836_T/TTGCA are linked together to form two haplotypes: Hap1_0/C/0 and Hap2_1/T/4.

FIG. 3A and FIG. 3B show the leaf angle phenotype comparison of inbred lines of different mutation types at the three variant sites in the ZmNAC16 intron and 3′-UTR region, where the Hap1_0/C/0 and Hap2_1/T/4 represent the two genotypes in FIG. 2, respectively; and FIG. 3C shows the expression analysis of the ZmNAC16 gene in pulvini of folded and unfolded leaves of the two inbred lines Hap1_0/C/0 and Hap2_1/T/4 at the V7 stage, where the ZmNAC16 gene is remarkably highly expressed in pulvini of unfolded leaves.

FIG. 4A shows the statistical analysis of corresponding phenotypes of the two allelic mutations of Hap1_0/C/0 and Hap2_1/T/4; and FIG. 4B shows the selection analysis of Hap1_0/C/0 and Hap2_1/T/4 in a maize breeding process, where colors corresponding to different allelic mutations are the same as in FIG. 4A and Hap1_0/C/0 is obviously artificially selected in the maize breeding process.

### DETAILED DESCRIPTION OF THE EMBODIMENTS

The present disclosure will be further described below in conjunction with specific examples, and the advantages and features of the present disclosure will become clearer from the description. However, these examples are only exemplary and do not constitute any limitation to the scope of the present disclosure. Those skilled in the art should appreciate that modifications and substitutions can be made to the details and forms of the present disclosure without departing from the spirit and scope of the present disclosure, but these modifications and substitutions fall within the protection scope of the present disclosure.

For the inbred lines used in the following examples, relevant information can be obtained and corresponding seeds can be applied from the “Chinese Crop Germplasm Resources Information System”.

### Example 1 Mutation Sites in the ZmNAC16 Intron and 3′-UTR Region Regulated a Maize Leaf Angle by Controlling an Expression Level of the ZmNAC16 Gene

1. Discovery of the Mutation Sites in the ZmNAC16 Intron and 3′-UTR Region

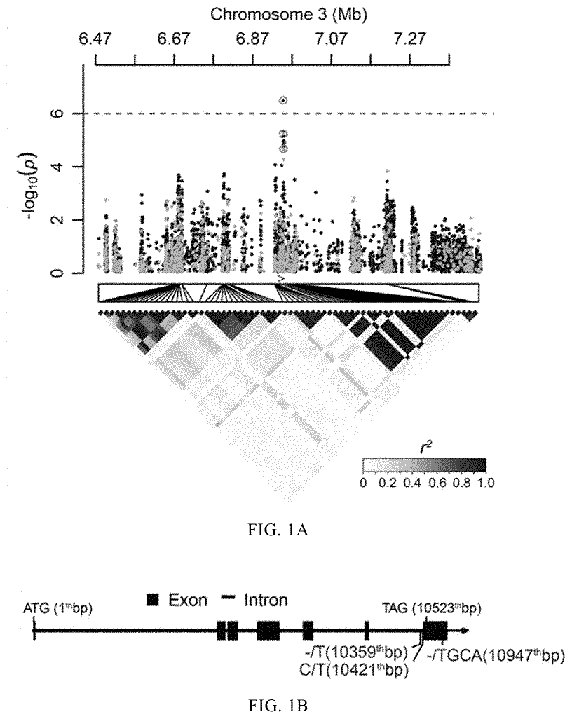

With deep (>10×) resequencing data of 350 maize inbred lines in combination with the published maize B73 V3 genome, 25,320,664 single-nucleotide polymorphism molecular markers (SNPs) and 4,319,510 indel polymorphism molecular markers (Indel) were discovered. These discovered molecular markers were used to estimate the population structure and genetic relationship of the 350 maize inbred lines, and then in combination with collected leaf angle phenotypes of 4 environments, GWAS was conducted. One SNP marker SNP_3_6945310_C/T and two Indel markers Indel_3_6945248_C/CT and Indel_3_6945836_T/TTGCA on chromosome 3 were found to be significantly associated with the maize leaf angle trait (FIG. 1). Further research showed that the three mutation sites were located in the intron and 3′-UTR (SEQ ID No. 2) region of ZmNAC16.

2. Acquisition of a Nucleotide Sequence of a Genomic Region where the Mutation Sites in the ZmNAC16 Intron and 3′-UTR Region were Located

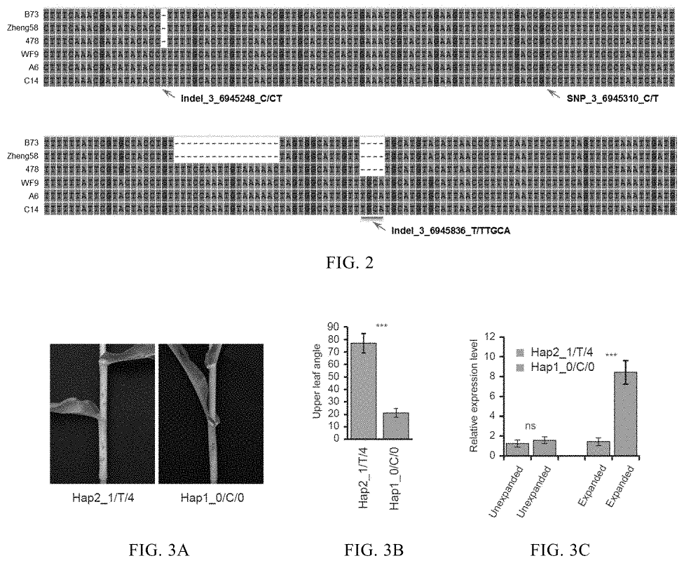

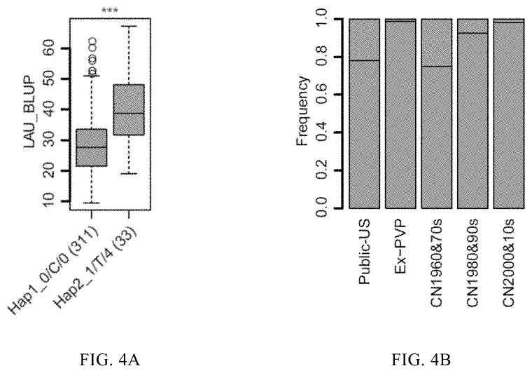

Specific amplification primers were designed based on the B73 V3 genome sequence of the region where SNP_3_6945310_C/T, Indel_3_6945248_C/CT, and Indel_3_6945836_T/TTGCA were located, and amplification was conducted for 6 different types of maize inbred lines to obtain the nucleotide sequence of this region and the accurate information about the mutation (FIG. 2).

3. Regulation of the ZmNAC16 Gene Expression by the Mutation Sites in the ZmNAC16 Intron and 3′-UTR Region

According to the genotypes at the three mutation sites SNP_3_6945310_C/T, Indel_3_6945248_C/CT, and Indel_3_6945836_T/TTGCA, the sequenced inbred lines were divided into Hap1_0/C/0 and Hap2_1/T/4 (FIG. 2 and FIG. 3). The two types of representative inbred lines (FIG. 2) were planted in the field, separately. When there were 7 fully unfolded leaves (V7 stage), pulvini (a part where a leaf and a leaf sheath are connected) of fully unfolded leaves (V7 leaves) and folded leaves (V5 leaves) were sampled and quick-frozen with liquid nitrogen. Pulvini of every 5 individual plants were mixed into a sample, and 3 biological replicates were set for each inbred line. Then RNA was extracted by the TRIzol method, and the expression level of the ZmNAC16 gene was detected using specific primers (SEQ ID No. 5 and SEQ ID No. 6). It was found that the ZmNAC16 gene was significantly highly expressed in the leaves of the Hap2_1/T/4 inbred line (FIG. 3), indicating that the mutation sites in the ZmNAC16 intron and 3′-UTR region could regulate the expression of the ZmNAC16 gene in maize leaves.

### Example 2 the Mutation Sites in the ZmNAC16 Intron and 3′-UTR Region were Subjected to Strong Artificial Selection in the Modern Maize Breeding Process

In the early stage of this experiment, 350 maize inbred line materials from different breeding periods in China and the United States were collected, including 163 breeding materials from the early period (Public-US) and the modern period (Ex-PVP) of the United States, and 187 main maize breeding materials from the early period (CN1960&70s), middle period (CN1980&90s), and current period (CN2000&10s) of China. The frequency distribution of the two genotypes Hap1_0/C/0 and Hap2_1/T/4 of ZmNAC16 in these materials was analyzed, and it was found that, with the advancement of the breeding period, the frequency of Hap1_0/C/0 had increased significantly in the maize breeding processes of China and the United States, indicating that the Hap1_0/C/0 genotype was subjected to strong artificial selection in the modern maize breeding process. Phenotypic analysis showed that Hap1_0/C/0 could significantly reduce the maize leaf angle, and it was consistent with the fact that the compact plant type is a key target for breeding of dense-planting-tolerant maize, which further confirmed that the mutation sites in the ZmNAC16 intron and 3′-UTR region were subjected to strong artificial selection in the modern maize breeding process.

What is claimed is:

1. A key DNA sequence for regulating a maize leaf angle, wherein the key DNA sequence has a polynucleotide selected from the group consisting of (a), (b), (c), and (d):
(a) a first polynucleotide sequence shown in SEQ ID No. 1;
(b) a second polynucleotide, wherein the second polynucleotide hybridizes with a complementary sequence of SEQ ID No. 1 under stringent hybridization conditions, wherein a protein encoded by the second polynucleotide still has a function of regulating the maize leaf angle;
(c) a third polynucleotide having at least 90% or more homology with the first polynucleotide sequence shown in SEQ ID No. 1; and
(d) a polynucleotide mutant obtained through deletion, substitution, or insertion of one or more bases on the basis of the first polynucleotide sequence shown in SEQ ID No. 1, wherein a protein encoded by the polynucleotide mutant still has the function or an activity of regulating the maize leaf angle.

2. A mutant of the key DNA sequence for regulating the maize leaf angle according to claim 1, wherein the mutant has a polynucleotide sequence shown in SEQ ID No. 2.

3. A recombinant expression vector, wherein the recombinant expression vector carries the key DNA sequence according to claim 1, or the recombinant expression vector carries a mutant of the key DNA sequence, and the mutant has a polynucleotide sequence shown in SEQ ID No. 2.

4. A molecular marker for regulating an expression of ZmNAC16 gene in a maize pulvinus, wherein the molecular marker comprises SNP_3_6945310_C/T, Indel_3_6945248_C/CT, and Indel_3_6945836_T/TTGCA.

5. A specific detection primer for detecting mutations of the molecular marker according to claim 4, wherein the specific detection primer has nucleotide sequences shown in SEQ ID No. 3 and SEQ ID No. 4.

6. Use of the key DNA sequence according to claim 1 in a regulation of the maize leaf angle, or in a cultivation of a high-yield maize variety or a dense-planting-tolerant maize variety, wherein the regulation of the maize leaf angle comprises reducing the maize leaf angle.

7. Use of the mutant according to claim 2 in a regulation of the maize leaf angle, or in a cultivation of a high-yield maize variety or a dense-planting-tolerant maize variety, wherein the regulation of the maize leaf angle comprises reducing the maize leaf angle.

8. Use of the molecular marker according to claim 4 in a regulation of the expression of ZmNAC16 gene in maize.

9. A specific primer pair for detecting ZmNAC16 gene, wherein the specific primer pair has polynucleotides shown in SEQ ID No. 5 and SEQ ID No. 6.

10. A method for cultivating a high-yield maize variety or a dense-planting-tolerant maize variety, comprising: using the key DNA sequence according to claim 1 or a mutant of the key DNA sequence to increase an expression level of ZmNAC16 gene in a maize pulvinus, wherein the mutant has a polynucleotide sequence shown in SEQ ID No. 2.
